(19) United States
(12) Patent Application Publication
(10) Pub. No.: US 2024/0278017 A1
(43) Pub. Date: Aug. 22, 2024

(54) METHOD OF TREATMENT OF STROKE WITH THIRD GENERATION SYNCHRONIZATION MODULATION ELECTRIC FILED AND A DEVICE THEREFOR

(71) Applicant: WR Biotech LLC, Tampa, FL (US)

(72) Inventors: Wei Chen, Tampa, FL (US); Ruisheng Liu, Tampa, FL (US)

(21) Appl. No.: 18/444,496

(22) Filed: Feb. 16, 2024

Related U.S. Application Data
(60) Provisional application No. 63/446,044, filed on Feb. 16, 2023.

(51) Int. Cl. A61N 1/36; A61N 1/05

(52) U.S. Cl. CPC A61N 1/36135; A61N 1/0504; A61N 1/3606; A61N 1/36171; A61N 1/36175

(57) ABSTRACT

A system and method for treatment of the stroke by electrically operating sodium-potassium (Na/K) pumps that consume one ATP to actively transport 3 Na⁺ and 2 K⁺ ions. The system and method provide a specially designed energy generating synchronization modulation electric field and utilizing a 4-electrode array to apply the field to the brain to effectively run the pump molecules in their physiological running mode by first consuming one ATP molecule to actively extrude 3 Na by exchanging 2 K ions; synchronizing the pump molecules down to individual steps throughout the pumping cycle; accelerating the moving ions to inject electric energy to the pumps; using electric energy to synthesize one ATP molecule at end of each pumping cycle; and modulating the pumping rate going up or going down to a pre-determined value. Consequently, the ATP molecules are recycled in the process of build-up ionic concentration gradient but not consumed. Electric energy becomes the driving force for the pumping cycle. As a result, the zero-consumption of ATP for the electrically modulated Na/K pumps controlled by the system and method of the present invention can save the residual limited ATP molecules in cells and maintain the living environment of brain, and therefore the brain functions regardless of the insufficient ATP supply.

### FIELD OF THE DISCLOSURE

The invention relates to devices and/or methods for treatment of stroke with the third-generation synchronization modulation electric filed (3^{rd} SMEF).

### BACKGROUND OF THE INVENTION

Health related complications in humans are on the rise due to various psychological, physiological, and behavioral factors. Numerous reasons contribute to these so-called “Lifestyle Diseases”, such as physical inactivity, unhealthy diet, smoking, alcohol etc., and are now one of the leading causes of deaths worldwide. These complications are responsible for procreating stroke, heart diseases, obesity, diabetes, and the like.

Stroke, for example, originates due to blockage of blood flow to the whole brain or any part thereof. It may also originate due to bursting of blood vessels in the brain that may lead to brain damage, mental disability or even death. The principle behind brain stroke includes diminished or lack of oxygenated blood supply to the brain which leads to death of cells inside the brain. There are two types of strokes: an Ischemic stroke and a Hemorrhagic stroke. Ischemic stroke occurs due to blood clots or any fatty deposition resulting in blockage of vessels of the brain responsible for supply of blood to brain. Hemorrhagic stroke, on the other hand, results from leakage or rupturing of the arteries, thereby building a strong pressure on brain cells due to bleeding from arteries and ultimately leading to cell damage. Out of these two strokes, Ischemic stroke is the most common one.

Ischemic cell injury may be defined as injury developed due to diminished flow of blood and the process is called hypoxia, which originates from interrupted supply of blood, lack of nutrients and toxic metabolites accumulation. Although more prominent in brain, however, other organs like kidney, heart, large intestine, lung, skeletal muscles, and liver are also susceptible, leading to multi-system organ failure. Depending upon the blood supply, ischemic cell injury may be reversible or irreversible. In reversible ischemia, the functions of the damaged cells are restored upon resumption of the interrupted blood supply to these cells, whereas, in irreversible ischemia, the cells are unable to restore the blood supply, as the reversibility threshold has already passed.

Other frequent causes of disease and death include ischemia reperfusion injuries such as stroke, myocardial infarction, and peripheral vascular disease. Ischemic reperfusion injury or reperfusion injury is caused when blood is restored to the tissues after ischemia attach leading to inflammation and oxidative damage, i.e., oxidative stress, due to sudden restoration of oxygen and nutrients supplied trough blood.

The cell membrane of all eukaryotes disseminates Na+/ K+ pumps, which actively transport sodium ions out of plasma membrane and potassium ions inside the plasma membrane, thus creating a chemical and an electrical gradient across the membrane. The generated gradients are responsible for maintaining membrane potential, secondary active transport of other solutes and maintaining cell volume etc. The Na+/K+ pump is situated at the cytosolic side of the outer membrane of the cell and functions as an electrogenic trans-membrane ATPase. Higher concentration of sodium is sustained extra-cellularly along with higher concentration of potassium intra-cellularly using concentration gradient via Na+K+-ATPase pump.

Acute phase ischemia-reperfusion injury is associated with diminished supply of oxygen leading to an alteration of ATP generation by mitochondrial oxidative phosphorylation. The brain ischemic reperfusion injury is mediated via complex I redox-dependent inactivation and a lack of oxygen leads to a loss of flavin mononucleotide (FMN) cofactor by mitochondrial complex I, making it inactive. The inactivation of mitochondrial complex I increases the production of reactive oxygen species (ROS) and reduces the production of energy from mitochondria causing extensive tissue destruction. The ATP production and pH level decreases intracellularly with increased duration of ischemia that leads to an accumulation of lactate followed by dysfunctioning of ATPase-dependent ion transport (Na+/K+ATPase) and ultimately leads to swelling, rupturing, and cell death.

Na/K pump is the most prevalent active transporter in the membrane of almost all kinds of cells. Na/K pump extrudes three Na ions out of the cell via the exchange of two K ions by consuming one adenosine 5′-triphosphate (ATP) for each pumping cycle to maintain the ionic concentration gradients and the potential difference across the cell membrane. Na/K pump is a unique energy converter converting the organic ATP hydrolysis energy to the inorganic electrochemical potential difference across the cell membrane so that many other membrane proteins can easily utilize the energy.

Since involving ion-movements across the cell membrane, Na/K pumps are sensitive to the membrane potential. In the last a few decades, significant efforts have been made to electrically control or manipulate the Na/K pumps. In early 1980's, Tsong and Tissee first showed that the kilo- and mega-Hz oscillating electric field can activate the Na- and K-transports, respectively for about 30% (1). Later, many models have been developed to explain the possible mechanisms in electrical activation of the pump molecules including resonance (2), Brownian motor (3), electronic transition (4), thermal noise (5), adiabatic process (6) and so on. However, now no practical technique is available that can effectively control the Na/K pumps, not mention saving ATP molecules.

Conventional arts disclose a technique known as Synchronization Modulation (SM), wherein an oscillating electric field is used to facilitate transportation of both molecules of Na+ and K+, alternatively. In first generation synchronization modulation (SM) technique, an oscillating electric field with an initial frequency close to the mean physiological turnover rate is used to synchronize the Na+/K+ pump molecules, and then modulate the pumping rate by changing the synchronization frequency. The turnover rate for Na+/K+ pumps using the technique has shown acceleration by many folds. However, the first-generation synchronization modulation technique can only synchronize the sodium and potassium transportations into their corresponding half cycles in the presence of ATP molecules.

Further still, such techniques are inefficient as they cannot efficiently maintain Na+/K+ pump activity in situations with insufficient ATP supply. Therefore, it cannot reduce infraction size, unable to reduce mortality rate, and unable to reduce ischemic injury or ischemia reperfusion injury nor able to maintain and improve prognosis and life quality.

As a result, there has been a need for methods and/or devices enabling the production of the 3^{rd} generation synchronization modulation electric field (SMEF). The technique can run the Na+/K+ pumps without ATP consumption, therefore effectively maintain and activate the pump functions even with insufficient ATP supply. Application of the said 3rd SMEF to brain of a subject can result in:
reduced infarction size after stroke;
reduced mortality;
improved prognosis and life quality;
maintained Na/K pump activity during ischemic conditions such as stroke;
reduced ischemic injury; and
reduced ischemia reperfusion injury.

### SUMMARY OF THE INVENTION

To treat the stroke of brain, it is important to retain the functions of the Na/K pumps regardless of the lack of ATP to keep the brain healthy. This section provides a general summary of the disclosure and is not a comprehensive disclosure of the full scope of all features.

In various embodiments, the present invention discloses a device and a method of treatment of strokes with related complications in human subjects using 3^{rd} SMEF (7) not only to save the residual limit ATP molecules by using electric energy to substitute ATP hydrolysis energy in fueling the pumping cycle but also activate the function of the Na/K pumps to maintain brain healthy environments. As a result, the brain can remain healthy to avoid ischemic stroke damage and complications.

The 3^{rd} generation synchronization modulation technique was developed from the 1^{st} (8) and 2^{nd} generations synchronization modulation technique which allow us to measure the real-time, semi-single pump currents. The first-generation synchronization modulation technique showed separated outward Na and inward K pump currents which ruled out the hypothesis that the pumping cycle reaches thermodynamic equilibrium, and able to modulate the pumping rate going up or going down accurately to a pre-determined value by gradually changing the synchronization frequency.

The second generation synchronization modulation technique revealed the real-time, semi-single pump currents measured from the physiological running condition showing the transient pump currents with the extremely short time course (less than 100 ms) as short as the membrane capacitance currents. The results confirmed the free and kinetical movements of the Na and K ions along a channel-like structure in the pump molecules. The extremely short time course of the transient pump currents represents that Na/K pump functions as a molecular machine with two power-strokes through a series of deterministic physical reactions instead of through a series of equilibrium stochastic chemistry reaction, and further rule out the possibility that ion-transports across the cell membrane are achieved by the protein conformational change in the transmembrane domain.

The free ion-movements along the channel-like structure also indicate that we will be able to inject electric energy to the pump molecules by acceleration of the ion-movements.

The 2^{nd} generation synchronization modulation technique further allows us to measure the real-time semi-single pump currents from the non-canonical pump model, such as the Na/Na exchange mode, where the same amount of the ions is transported across the cell membrane without ATP consumption. Again, the transient, separated inward and outward pump currents show the extremely short time course as short as the membrane capacitance currents with the same magnitude. The transient pump currents, especially the inward pump currents as transient as the outward pump currents with the same magnitude indicate that the ATP hydrolysis energy drives the Na ions kinetically moving out of the cell to build up ionic concentration gradient, and then, the potential energy is fully converted to the kinetic energy of the downhill Na ions moving into the cell. Based on the energy conservation law, hydrolysis of one ATP molecule releases 550 meV energy (9), synthesis of one ATP molecule requires at least the same amount of energy. Therefore, the kinetic energy carried by the downhill Na ions must be the driving force for the ATP synthesis, and the site of ATP synthesis must me in the intracellular loop domain.

Clearly, Na/K pumps have dual energy-convert features, converting ATP hydrolysis energy into the electrochemical potential across the cell membrane by using the kinetic energy of ions as the media, and utilizing the electrochemical potential energy to synthesize ATP again using the kinetic energy of ions as the media.

By utilizing the dual energy-convert features of the Na/K pumps in both the physiological running mode and the non-canonical pumping mode, and by introducing the concept of electronic synchrotron accelerator in physics to the biological system, the 3rd generation synchronization modulation electric field was developed that can use electric energy to substitute ATP hydrolysis energy in fueling the pumping cycle. Briefly, Na/K pumps first run in the physiological condition consuming ATP hydrolysis energy to transport 3 Na and 2 K ions against the electrochemical potential difference to build up ionic concentration gradients. Meanwhile, an oscillating electric field is applied to the cell membrane to synchronize the pump molecules down to the individual steps in the pumping cycle and inject electric energy to the pump molecules by accelerating the ion movements. Due to the energy transfer function of the pump molecules, the energy will eventually be accumulated on the inward 2 K ions. Then, switching the pumps to the non-canonical pumping mode, the kinetic energy of 2 K ions will be used as the energy course to re-phosphorylate ADP to form ATP. As a result, one ATP molecule is first hydrolyzed to drive 3 Na and 2 K ions actively transported across the cell membrane to build up ionic concentration gradient, and then re-synthesized by using electric energy. As a result, ATP molecules are recycled but not consumed. Electric energy becomes the driving force for the pumping cycle.

As a result, even when ATP molecules have been thoroughly washout from the cells, and in the presence of blocker of ATP synthesizers in mitochondria, the 3^{rd} generation synchronization modulation electric field can still generate the outward Na and inward K pump currents, indicating the net ATP-consumption is zero.

The present invention involves a computer-controlled device that readable medium storing a set of instructions configured for being executed by at least one processor for performing a method for controlling Na/K pumps, the method comprising: applying a well-designed oscillating electric field to synchronize the ion-pumping in half-cycle and the ion-extrusion half-cycle of the Na/K pumps, wherein the ion-pumping in half-cycle is in a negative half-cycle and the ion-extrusion half-cycle is in a positive half-cycle of the oscillating electric field, applying an activation overshoot electric pulse at the start of the negative half-cycle and at the start of the positive half-cycle of the oscillating electric field, and applying an energy-trap overshoot electric pulse at the end of the negative half-cycle and at the end of the positive half-cycle of the oscillating electric field, wherein the activation overshoot electric pulse, the energy-trap overshoot electric pulse and the electric field plateau in between the activation overshoot electric pulse and the energy-trap overshoot electric pulse synchronize the Na/K pumps down to individual steps throughout the running cycle, wherein the outward transporter current is restricted at the start of the activation overshoot electric pulse in the positive half-cycle and the inward transporter current is restricted at the start of the activation overshoot electric pulse of the oscillating electric field, and synthesize of one ATP molecule during each running cycle of the Na/K pump. The generated ATP molecule compensates for the ATP consumed in actively transporting ions. As a result, the ATP net-consumption of the electrically modulated Na/K pumps is significantly reduced, theoretically to zero.

In a various embodiment, the present invention provides a system and method for application of the 3^{rd} generation synchronization modulation techniques to the brain by synchronizing the Na/K pumps, down to individual steps in the running cycle; and driving the active ion transports in the physiological running conditions to actively transports ions across the cell membrane by consuming one ATP, and simultaneously injecting electric energy to the pumps to synthesize one ATP for each pumping cycle. As a result, the generated ATP molecules compensate for the ATP consumed in actively transporting ions across the cell membrane. The net ATP consumption of the electrically synchronized Na/K pumps is significantly reduced, theoretically to zero, and then, gradually modulate the Na/K pumps or reduce the pumping rate to a pre-determined turnover rate by progressively reducing the synchronization frequency of the Na/K pumps in a stepwise pattern.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows pump currents measured from the dialyzed pump molecules by fully removing Na ins and in the absence of ATP molecules.

FIG. 2 shows pump currents measured from the physiological running condition showing the real-time, semi-single pump currents having the transient, separated outward Na and inward K currents with the extremely short time course.

FIG. 3 illustrates the real-time, semi-single pump currents measured from the non-canonical Na/Na exchange mode. The transient inward pump currents with the same magnitude and duration as the outward pump currents indicate that potential energy across the cell membrane is fully converted to the kinetic energy carrying by the downhill inward Na ions moving into the cell to facilitate ATP synthesis.

FIG. 4 shows Na/K pump currents measured by the traditional single pulse, 2^{nd} generation and 3^{rd} generation synchronization electric field. After removing ATP molecules, the unchanged pump currents generated by the 3^{rd} generation synchronization electric field with the magnitude of activation overshoot electric pulse over 90 mV prove the zero-consumption of the ATP in active transport Na and K ions.

FIG. 5 illustrates the waveform of the forward modulation electric field in three phases, synchronization (A), modulation (B), an maintenance (C), and the pump currents generated by the forward synchronization modulation electric field (D).

### DETAILED DESCRIPTION OF THE INVENTION

In various embodiments, the present invention provides a method to maintain the brain fresh or healthy by applying the 3^{rd} generation synchronization modulation electric field which can not only utilize electric energy to substitute ATP hydrolysis energy in fueling the pumping cycle but also accelerate the activity of Na/K pumps to a specific pumping rate to fit the brain requirements. As a result, the brain can be maintained fresh and healthy for longer time regardless of the residual limited ATP molecules.

In the following detailed description, the underlying mechanisms involved in development of the 3^{rd} generation synchronization modulation electric field of the Na/K pumps is presented based on the dynamics of the pump molecules. The parameters of electric field including the detailed waveform, magnitude and duration for the energy trap, activation overshoot, and plateau, initial and final frequency as well as the step-change are specially designed for the pump molecules in brain tissues. The design is not only to effectively control the pump functions but also to avoid side effects on other membrane proteins to maintain activity of the brain. The specific electrode array is soled developed based on the structure, orientation, dimension of the brain. The purpose of the invention is to maximally maintain healthy or fresh of the brain.

Na/K ATPases, or Na/K pump, is a prevalent active transporter in almost all kinds of cells. In operation, the pump extrudes 3 Na ions by exchanging 2 K ions to build up Na and K concentration gradients and the potential difference across the cell membrane, thereby providing the critical environment for living cells. The pump generated electrochemical potential difference across the cell membrane including ionic concentration gradient and the membrane potential difference is the source for many voltage-dependent membrane proteins. For example, various voltage-dependent or voltage-gated ion channels utilize the membrane potential to generate the action potential and propagate the action potential along the cell membrane. Another example is

Na/H exchangers using the Na concentration gradient as the energy source to transport H ions in influencing pH value. The ionic concentration gradients also play a significant role in controlling the cell volume and homeostasis. Therefore, reducing the functions of Na/K pumps to a specific value to fit the decreased metabolic reactions and saving the residual limited ATP concentration but maintain the necessary ionic concentration gradient and the potential difference across the cell membrane for the living cells is critical to the brain.

Therefore, Na/K pump is a unique energy converter converting the organic ATP hydrolysis energy to the inorganic energy of electrochemical potentials across the cell membrane, the energy source for many other membrane proteins including ion-channels, various pump molecules, and secondary active transporters.

Because Na and K ions are transported across the cell membrane against the concentration gradients, the pump functions are sensitive to the membrane potential. Therefore, it is possible to electrically influence the pump functions. However, in order to electrically fuel the pumping cycle without ATP consumption, we have to understand the mechanisms, especially the dynamics in the pumping cycle, or how the ATP hydrolysis energy drive the pumping cycle to actively transport Na and K ions.

Since being discovered in 1950s, Na/K pumps have been well studied. Based on the Pos-Albert model (10, 11), pumping cycle consists of a series of stochastic biochemical reactions through a random walk. The pumping cycle reaches the thermodynamics equilibrium without power-stroke in the pumping cycle. The protein conformational changes among E1 and E2 transport 3 Na and 2 K ions across the cell membrane. One of the pieces of evidence is that the measured pump currents show the small, static, net-outward currents (12-16).

Later, two-access-channel model was developed which allocates the protein conformational changes deeply inside the cell membrane, so called the “binding pocket”, sandwiched by two access channels connecting the internal and external solution (17) The evidence comes from the study of the dialyzed pumps where all the K ions (both the internal and external) were eliminated so that the pump molecules can only react with Na ions. The results showed that a negative electric stimulation pulse can generate forward pump currents. Once the stimulation is over, the backward pump currents with the same magnitude and time course as the forward pump currents were generated (18-20) These are typical feature of the displacement current indicating that the channel-like structure is somehow blocked deeply in the cell membrane.

Currently, little is known about the dynamics of the pumping cycle, i.e., how the ATP hydrolysis energy released in the cytoplasmic loop drives the pumping cycle and generating the protein conformational change to active transport Na and K ions.

To better understand the dynamics of the Na/K pumps, the inventor conducted energy analyses for the pumping cycle based on the Energy Conservation Law. The Na and K concentration gradients across the cell membrane are equivalent to the potential difference of Na and K ions as +60 mV and −90 mV, respectively (21). It is well-known that Na/K pumps run well in a wide range of membrane potentials, about ±100 mV with respective to the membrane resting potential at about −90 mV (22). At the membrane potential of −123.3 mV, which is in the range of pump running potentials, the energy required to extrude 3 Na ions is about 550 meV (=3e (123.3+60) mV), the same as the energy released from hydrolysis of one ATP molecule (23). All the ATP hydrolysis energy must be used to drive out 3 Na ions against the potential difference. No energy is left for the protein conformation change. This simple but fundamental energy analysis suggests that ion-movements across the cell membrane cannot be achieved by the protein conformational changes. ATP hydrolysis energy must somehow directly drive 3 Na ions out of the cells against the potential difference, and therefore, Na/K pump should have a channel-like structure in the transmembrane domain. This is similar to send a ball to the top of a building with minimum energy, the best way is the kick up the ball.

This is consistent with the previous energetic studies of the ATP hydrolysis. ATP hydrolysis energy is not released when the chemical bound is broken from ATP to ADP*Pi, but at the time the inorganic phosphate (Pi) is repelled by the electrostatic force physically moving away from ADP outside the ATPase active sites. The biochemical ATP hydrolysis energy is converted to the kinetic energy carried by the cleaved Pi (24-27) to drive 3 Na ions freely and kinetically moving out of the cell against the electrochemical potential difference through the channel-like structure.

The channel-like structure hypothesis is consistent with the previous studies of channel configuration (28-31). The PTX-treated pump molecules exhibit the channel functions allowing ions to pass through (32,33). Studies of the reactivity of each amino acid in different transmembrane helices using the substituted cysteine accessibility method (SCAM) revealed a single unbroken cation pathway through the pump-channel (34-36).

As a result, Na/K pump should have separated, transient outward Na and inward K currents. Due to the free and kinetical movements of the ions along the channel-like structure, the moving speed must be fast to have enough energy to overcome the potential difference. Therefore, the time required for ions to move across the cell membrane should be extremely short showing transient pump currents. Due to the Na and K ions sequentially moving across the cell membrane, Na- and K-pump currents must be separated. All these are different from the measured pump currents showing the static, net-outward currents.

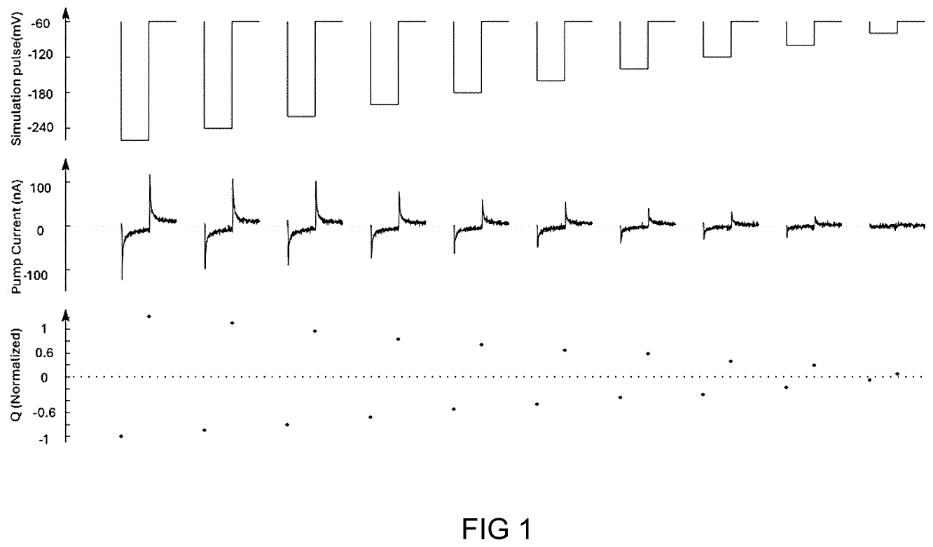

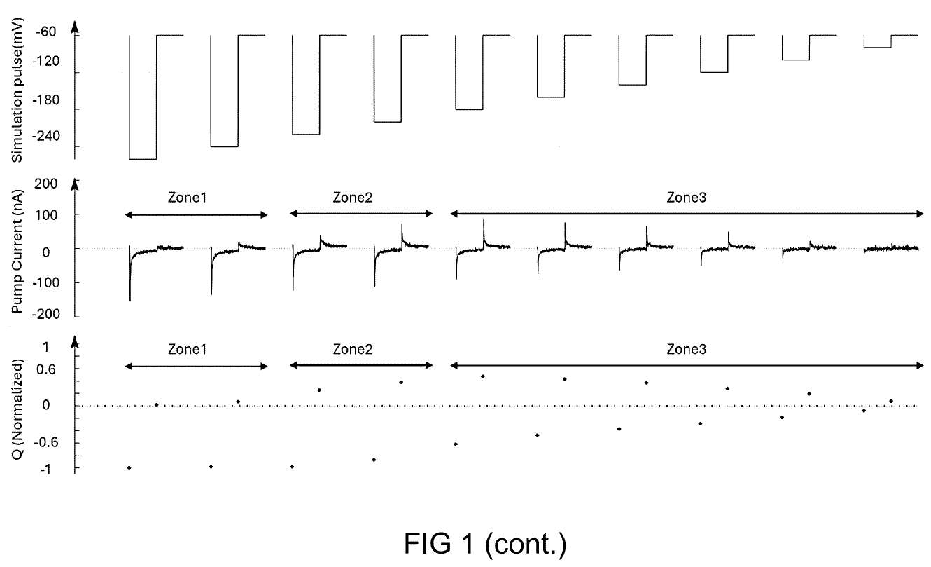

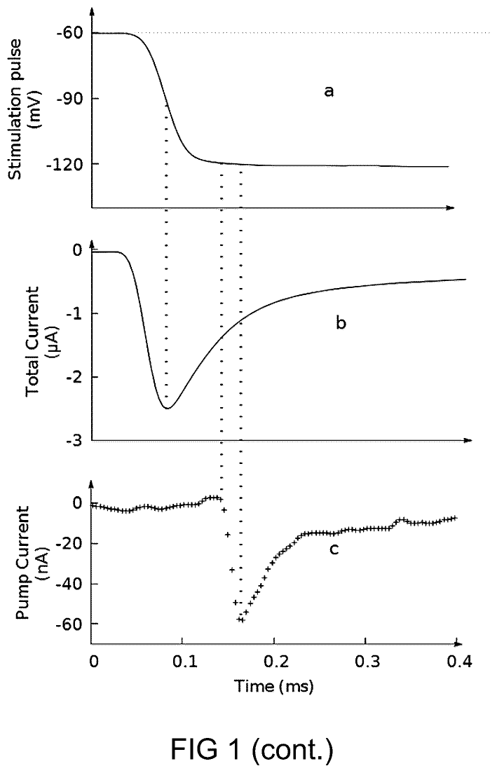

As shown in FIG. 1, for the dialyzed pump molecules, a stimulation pulse can generate uni-directional transmembrane pump currents. Duration of the pump currents is extremely short similar as the membrane capacitance currents. These results indicated the free movements of the K ions into the cell. Due to without ATP molecules, the moving-in K ions are not achieved by the protein conformational changes.

In contrast to the voltage-gate ion-channels all open at the same time responding to the membrane potential changes, Na/K pumps have random pumping pace and different pumping rate. Details of the pump currents will be inevitably merged into the current summation showing the static, net-outward pump currents. To observe the details of pump currents, the inventor started to synchronize the pump molecules by an oscillating electric field. For the first-generation synchronization modulation electric field, the pump molecules are synchronized to the Na- and K-transports, therefore, showing the separated outward Na and inward K pump currents but having static values.

Due to the opposite voltage-dependence, the field in the positive half-cycle facilitates the Na-transport but hindering the K-transport, so that the positive half-pulses are the Na-transport favorite half-pulse and K-transport unfavorite half-pulse. The negative half-pulse activates K-transport but deactivating the Na-transport so that they are the K-transport favorite half-pulse and the Na-transport unfavorite half-cycle. As the field oscillating, Na-transports from all the pump molecules will eventually fall into the positive half-cycle, and the K-transports into the negative half-cycle, respectively. Consequently, all the pump molecules run at the same pumping rate as the field oscillating frequency.

The oscillating electric field has symmetric waveform which can effectively synchronize all the pump molecules in two hemispheres. The field-induced membrane potentials on two hemispheres are always opposite. Any membrane potential change will facilitate the pump molecules in one hemisphere but inevitably hinder the pump molecules in another hemisphere. Under the symmetric oscillating electric field, pump molecules in two hemispheres can be synchronized into two groups with 180 phase-shift. In other words, one half-cycle of the oscillating electric field will facilitate the Na-transport of the pump molecules in the first hemisphere and the K-transport of the pumps in the second hemisphere. Then, another half-cycle of the oscillating electric field will facilitate the K-transports of the pump molecules in the first hemisphere and the Na-transport of pumps in the second hemisphere. As a result, all the pump molecules in two hemispheres are synchronized to the same pumping cycle.

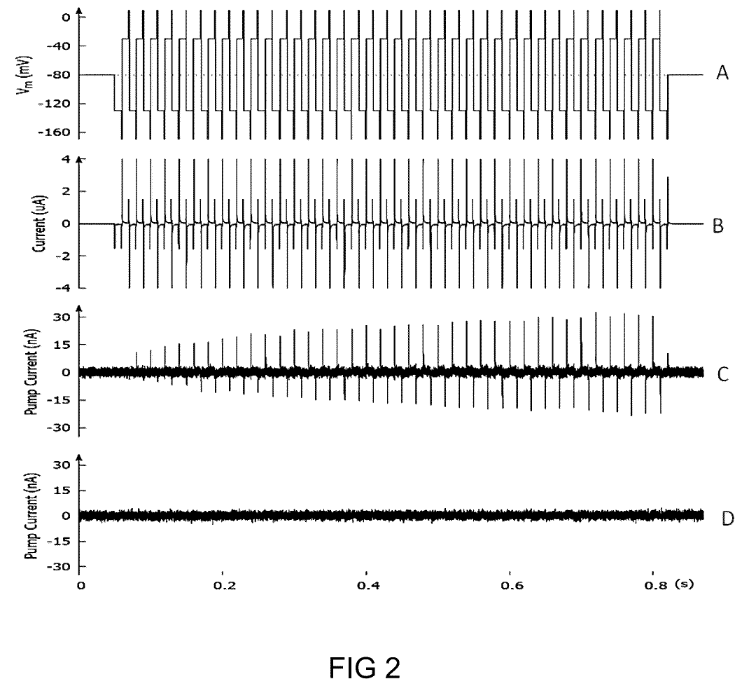

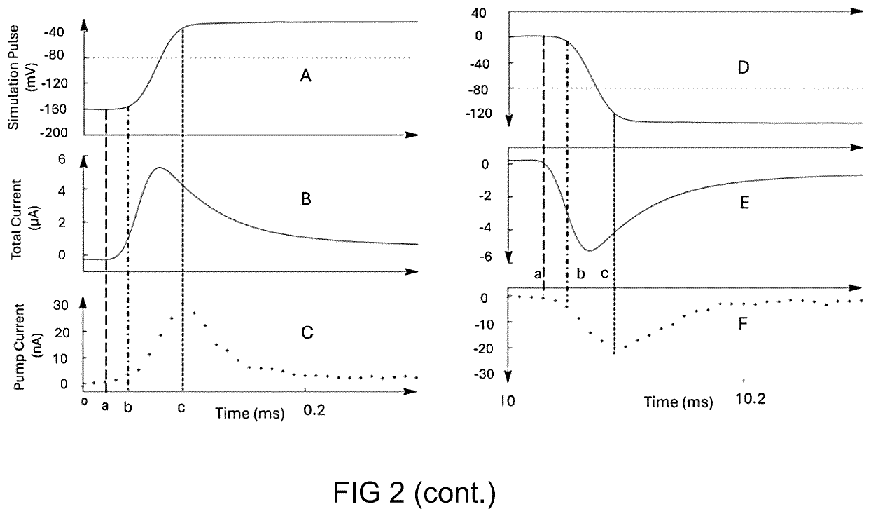

Later, the inventor improved the technique as the 2^{nd} generation which can synchronize the pump molecules down to the individual steps throughout the pumping cycle, resulting in the real-time, semi-single pump currents. As shown in FIG. 2. Under the 2^{nd} generation synchronization electric field, the synchronized pump molecules in physiological running condition generated the separated, transient outward Na and inward K pump currents having the extremely short time course as short as the membrane capacitance currents. To confirm the pump currents and the membrane capacitance currents having the similar duration, we purposely increased or decreased the gain of voltage-clamp to shorten or expand the duration of transmembrane (mainly the capacitance) currents. Regardless of the changes in gain, they always have a similar duration. The results indicate the ions freely and kinetically moving across the cell membrane through a channel-like structure.

Based on these and other studies, the inventor built up a dynamic model of the Na/K pumps expressed as follows: ATP hydrolysis energy is converted to the kinetic energy of the inorganic Pi to drive 3 Na ions out of the cell. Na ions carry the ATP hydrolysis energy as the initial kinetic energy freely and quickly moving against the electrochemical potential difference along a channel-like structure. At end of the channel, the energy left on 3 Na ions is transferred at the loop domain to the K-transport driving 2 K into the cell along the channel. Similarly, whatever energy is left on the 2 K ions is also transferred to the Na-transport to compensate for the energy deficient in Na-transport. As a result, the limited ATP hydrolysis energy can drive the Na/K pumps running in a wide range membrane potential. Kinetic energy of ions plays a significant role in the pumping cycle.

In addition to the normal physiological running mode consuming ATP to actively transport Na and K ions, Na/K pump have several non-canonical modes, such as K-K exchange mode (37) and Na/Na exchange mode (38) where the same amount of ions are transported back and forth across the cell membrane without ATP consumption. Briefly, in the first half-cycle, ATP hydrolysis energy drives 3 Na ions out of the cell to build up potential energy difference, while in the next half-cycle, the Na potential energy drives 3 Na ions downhill into the cell, where the kinetic energy carried by the 3 Na inward ions is the energy source for the ATP synthesis.

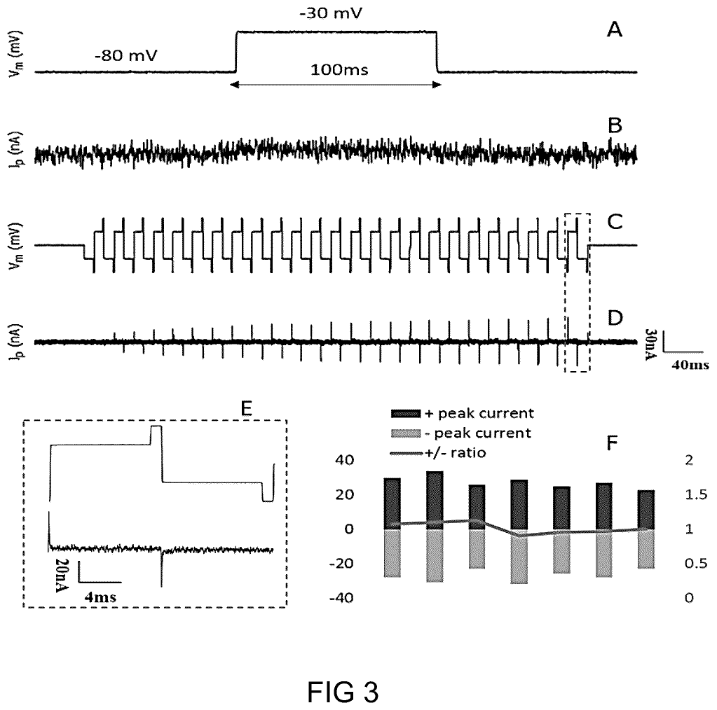

The synchronized pump currents at the Na/Na exchange mode showed transient outward and inward pump current with the same magnitude and duration (FIG. 3). The results indicate that similar as the ATP driving Na ions freely and kinetically out of the cell, the same amount extracellular Na ions quickly move into the cell, freely and kinetically, where the potential energy is converted as the kinetic energy of ions to synthesize ATP molecules. This is similar as the ATP synthesizers, such as bacteriorhodopsin (39-40) and F0F1-ATPases (41-42), proton potential energy is transformed to the mechanic energy of the down-stream protons to rotate the F0 domain in F0F1 ATPase to synthesize ATP.

The strategy in development of the 3^{rd} generation synchronization modulation is to utilize the dual energy conversion features by alternatively running the normal pumping mode and the non-canonical model and introducing the concept of the electronic synchrotron accelerator in physics to the biological system to inject energy to the pump molecules by accelerating the moving speed of ions. Initially, Na/K pumps are synchronized to the physiological mode consuming ATP to drive 3 Na and 2 K ions. Simultaneously, the electric field will be used to first synthesize the pumping molecules down to individual steps throughout the pumping cycle and then accelerate the ion-movement to inject energy to the pump molecules. At end of the pumping cycle, switching the pumps to the non-canonical mode so that the kinetic energy carried by the 2 K ions kick out the Pi into the solution. Due to large molecular weight of nucleotide, the diffusion constant of ADP is 3 orders smaller less than ions. The ADP molecules released at beginning of the pumping cycle cannot move far away. When Pi carrying high energy hits the ADP, they form the ATP. As a result, the ATP consumption is significantly reduced, theoretically to zero.

The present invention, the 3^{rd} generation synchronization modulation technique, provides a method or a process to electrically fuel Na/K pumps and control the turnover rate of the pumping cycle, to a pre-determined value with zero consumption of ATP.

For each half-cycle of the oscillating electric field, there are three sections, short activation overshoot electric pulse and the energy trap overshoot electric pulse sandwiching the plateau with a wide duration and low magnitude. Once the Na- and K ion-transports falling into the favorite positive and negative half-pulses, respectively, the plateaus will activate the corresponding transports moving the Na and K pump currents towards to the front of half-pulses, in the consecutive half-pulses. Once falling into the energy trap overshoot pulse in the front un-favorite half-pulse, the ion-transports will be inhibited until the pulse changing its polarity. Therefore, the steps of Na- and K-movements through the channel-like structure, or the pump currents will be restricted into the beginning of the favorite half-pulses, respectively.

Duration and magnitude of two overshoot electric pulses and the plateaus are well-designed. For the energy-trap overshoot, it must be high enough to block the un-favorite ion-transport. Duration will depend on how fast we want the pump molecules being synchronized.

The wider the energy-trap overshoot, the faster the pump molecules being synchronized, or the fewer oscillating pulses are required to synchronize the pump molecules. However, the wider the overshoot, the higher the chance to influence other voltage-dependent membrane proteins. Duration of the activation overshoot is only a few hundred microseconds long enough to cover the pump currents. Magnitude is calculated based on the mechanisms involved in the ATP synthesis and the minimum energy required to synthesize one ATP.

The present invention, the 3^{rd} SMEF consists of three phases. Phase 1 is to synchronize the pump molecules down to the individual steps throughout the pumping cycle and injecting electric energy to the pumps to synthesize ATP. Phase 2 is to modulate the pumping rate of the synchronized pump molecules gradually to a pre-determined value by changing the synchronization frequency in a step wise pattern. Phase 3 is to maintain the pumping rate at the target value for certain period based on the clinical requirement.

The 3^{rd}-SMEF has been specifically designed to avoid side effect on the cell membrane, or the phospholipid bilayer and other membrane proteins. For example, to avoid changing integrity of cell membrane the field-induced membrane potentials are all in physiological range, much lower than the thresholds of the membrane electroporation and the protein denature. Duration for both overshoot pulses is short not enough to open the voltage-gated ion channels, including the Na-channels having the fastest electric response, or affect other membrane proteins. The waveform of the electric field was specifically designed for Na/K pumps, transporting cations to the opposite directions, and the 50 Hz frequency is comparable to the turnover rate of the Na/K pumps, far away from other pumps, such as Ca2+ pump of 500 Hz.

The present invention may be embodied on various computing platforms that perform actions responsive to software-based instructions and most particularly on touchscreen portable devices. The following provides an antecedent basis for the information technology that may be utilized to enable the invention.

The computer readable medium described in the claims below may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any non-transitory, tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. However, as indicated above, due to circuit statutory subject matter restrictions, claims to this invention as a software product are those embodied in a non-transitory software medium such as a computer hard drive, flash-RAM, optical disk, or the like.

Program code embodied on a computer readable medium may be transmitted using any appropriate medium, including but not limited to wireless, wire-line, optical fiber cable, radio frequency, etc., or any suitable combination of the foregoing. Computer program code for carrying out operations for aspects of the present invention may be written in any combination of one or more programming languages, including an object-oriented programming language such as Java, C#, C++, Visual Basic or the like and conventional procedural programming languages, such as the “C” programming language or similar programming languages.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

In accordance with an aspect of the present invention, the method of treatment comprises marking a number of spots, preferably four or more, distributed on the skull of a subject and drilling holes at the marked spots through the skull of the subject. Further, electric pins are placed in the drilled holes and the third generation SMEF is applied throughout the whole brain via a series of pin electrodes for maintaining the Na+/K+ activity during ischemic conditions such as stroke. Wherein the pin electrodes are designed as a specific array to be inserted in the skull of the subject.

In accordance with another aspect of the present invention, the method of treatment further comprises applying the 3^{rd} SMEF to brain with specific methods, including electrode design, the way of applying the electrodes to the brain, and duration of the field application.

In accordance with yet another aspect of the present invention, the method of treatment further comprises applying the 3^{rd} SMEF with optimized parameters of the electric field specifically fit the brain stroke, including magnitude and duration for each section of waveforms, the field initial and final frequencies, duration for each phase, and the modulation modes, forward or backward modulation.

In accordance with another aspect of the invention, a device for applying third generation SMEF to the brain of a subject is disclosed.

In accordance with further aspect of the invention, the device as disclosed advantageously delivers the third-generation synchronization modulation electric filed throughout the whole brain to improve brain function in the subject.

The disclosed method and the device significantly reduce both ischemic injury and ischemia reperfusion injury. The present invention effectively reduces infarction size after stroke, consequently, decreases mortality, and improves prognosis and life quality of the subject.

Additional aspects and advantages of the present disclosure will become apparent to those skilled in this art from the following detailed descriptions, wherein only illustrative embodiments of the present disclosure are shown and described. The present disclosure is capable of other and different embodiments, and its several details are capable of modifications in various obvious respects, and its several details are capable of modifications in various obvious respects, all without departing from the disclosure. Accordingly, the drawings, description and examples in this summary are intended for purposes of illustration only and are not intended to limit the scope of the present disclosure.

The embodiments herein and the various features and advantageous details thereof are explained more fully with reference to the non-limiting embodiments that are illustrated in the accompanying drawings and the following description. Numerous variations, changes and substitution may occur to those skilled in the art without departing from the invention. Various alternatives to the embodiments of the present disclosure herein may be employed.

At the outset, for ease of reference, certain terms used in this application and their meanings as used in this context are set forth. To the extent a term used herein is not defined below, it should be given the broadest definition persons in the pertinent art have given that term as reflected in at least one printed publication or issued patent. Further, the present techniques are not limited by the usage of the terms used in application, as all equivalents, synonyms, new developments, and terms or techniques that serve the same or a similar purpose are considered to be within the scope of the present claims.

The articles “a” and “an” as used herein mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

The present invention relates to a device and/or a method of applying the third-generation synchronization modulation electric filed (3^{rd} SMEF) to brain of a subject for treatment of ischemic stroke. The device, in an embodiment, facilitates insertion of a plurality of pin electrodes through the skull for applying third generation SMEF throughout the whole brain or the specific area based on the requirements via the pin electrodes. The method, in an embodiment, includes applying the synchronization modulation electric field through a series of pin electrodes designed as a specific array to be inserted in the skull of the subject at the predefined locations.

The method, in another embodiment, includes applying the 3^{rd} SMEF to the brain with specific methods, including electrode design, the way of applying the electrodes to the brain, and duration of the field application.

The method, in yet another embodiment, includes applying the 3^{rd} SMEF with optimized parameters of the electric field specifically fit the brain stroke, including magnitude and duration for each section of waveforms, the field initial and final frequencies, duration of each phase, and modulation modes. The present invention improves brain function in the subject. In particular, the method as disclosed helps maintenance of Na+/K+ pump activity during ischemic conditions such as stroke thus significantly reducing ischemic injury and ischemia reperfusion injury. Further, the invention effectively reduces infarction size after stroke, consequently, decreases mortality, and improves prognosis and life quality of the subject.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### EXAMPLES

### Example 1: Methods for Transient Middle Cerebral Artery Occlusion (MCAO)

The below steps describe the application of SMEF on a mouse model. The method steps help understanding the effect of third generation of SMEF on a mouse model includes the following:
Anesthetize an 8-12-week-old mouse (25-30 g) with isoflurane.
Place the mouse in the supine position on a heating pad. Insert a rectal probe, and monitor and maintain body temperature between 36.5-37.5° C.
Shave the fur on the ventral neck region. Disinfect the surgical site using three applications of 70% ethanol.
Make a 1 cm long midline incision on the neck. Use retractors to expose the surgical field and identify the right common carotid artery (CCA), external carotid artery (ECA), and internal carotid artery (ICA). Carefully dissect the arteries free from surrounding nerves and fascia.
Dissect the external carotid artery (ECA) further distally and coagulate the external carotid artery (ECA) and its superior thyroid artery (STA) branch using a bipolar coagulator. Cut the external carotid artery (ECA) and superior thyroid artery (STA) at the coagulated segment.
Loosely tie two 8-0 silk sutures around the external carotid artery (ECA) stump. Apply a vascular clamp (Fine Science Tools) at the bifurcation of the common carotid artery (CCA) into the external carotid artery (ECA) and internal carotid artery (ICA).
Make a small incision at the end of external carotid artery (ECA) stump with Vannas style spring scissors (Fine Science Tools). Measure and record the length of a 5-0 monofilament suture rounded at the tip. Insert the suture into the incision and advance to the clamp. Tighten the two silk sutures around the lumen just enough to secure yet preserve mobility of the in-dwelling monofilament suture.
Remove the clamp from the bifurcation. Gently advance the monofilament suture from the lumen of the external carotid artery (ECA) into the internal carotid artery (ICA) for a distance of 9-10 mm beyond the bifurcation of common carotid artery (CCA) to occlude the origin of Middle Cerebral Artery (MCA) for 2 hours. Then remove the suture.
Suture the incision on the neck and place the mouse in a 35° C. nursing box to recover from anesthesia, and return it to the cage. It generally takes 5-10 min for the mice to recover from anesthesia.
Twenty-four hours after the induction of Middle Cerebral Artery Occlusion (MCAO), euthanize the mouse and collect the brain. Slice the brain coronally into four 2-mm slices and quantify the extent of infarction area.

### Example 2: Application of Synchronization Modulation Electric Filed (SMEF)

Shave the top of the head of a subject.
Drill 6 holes of diameter are about 0.5 mm evenly distributed on the top of head of the subject.
Put electrode pin in the drilled holes.
Start SMEF for 2 hours during MCAO, then stop SMEF and remove electrodes.
Observation of infarction size at 24 hours after MCAO.

### DETAILED DESCRIPTION OF THE FIGURES

FIG. 1 illustrates the pump currents measured from the dialyzed pump molecules. Na ions in both the internal and external solutions are fully eliminated, and ATP concentration was significantly reduced by at least five times washout. Upper traces in both upper and middle sections show a series of stimulation pulses from −20 to −200 mV at the holding potential of −60 mV. Middle traces show the generated pump currents. For the upper section, the intra- and extra-cellular K concentration was 140 and 8 mM, respectively, as the previous studies (43). The stimulation pulse drives the forward pump current indicating K ions moving into the protein. Once the stimulation pulse was over, the backward pump currents were generated with the same magnitude and time course, representing the ions moving back out of the pump molecules, or the channel-like structure is blocked deeply inside the cell membrane. The results are the same as those obtained in previous study (FIG. 2 in ref.(43)). The displacement features of the pump currents are considered as evidence of the extracellular access-channel in the two-access-channel model.

For the lower section, only the extracellular K concentrations are increased to 40 mV which is still much lower than the intracellular K concentration. The same stimulation pulses generated uni-directional inward transmembrane pump currents.

Lower section draws the stimulation pulse, the corresponding total transmembrane (mainly capacitance) current, and the transient pump currents at the same timescale. Duration of all the two-way displacement currents and the uni-direction transmembrane pump currents have the same time course of about 100 μs as the membrane capacitance currents. The results suggest that no protein conformational change is involved in the ion movement in the pump molecules.

FIG. 2 Upper panel is 39 oscillating pulses from the 2^{nd} generation synchronization electric field. To identify the pump currents, a pre-pulse (not shown) with the same waveform of the oscillating electric field was also applied to the cell membrane. The induced currents function as a template current to be subtracted from the currents generated by each individual oscillating pulses (middle panel, in μA). The difference is the synchronized pump currents as shown in the lower panel (in nA).

For the first a few oscillating pulses, there were no pump currents at all because the pump molecules remained at randomly paced. As more and more the Na/K pumps were synchronized, the separated transient outward Na and inward K currents were generated responding to the start of the activation overshoots, and gradually increased until saturation. Magnitude of the transient pump currents is much (tens times) larger than the net outward pump currents measured from the random paced pumps. The outward Na currents over the inward K currents is about 3:2, reflecting the stoichiometric ratio of Na/K pumps. Both the outward and inward pump currents are transient with the time course about 100 μs, the same as the membrane capacitance currents.

The transient pump currents are redrawn in the lower section (C and F) with the total transmembrane currents (mainly the capacitance currents, B and E) and the membrane potential changes (A and D) in the same coordinate. Both the membrane capacitance currents, and the Na and K pump currents are restricted to the rising phase of the half-pulses having the similar duration.

The transient pump currents with the same time course as the membrane capacitance currents further prove the ions moving across the cell membrane without protein conformational change involved, or the Na/K pump has a channel-like structure.

FIG. 3 The real-time, semi-single pump currents measured from the non-canonical Na/Na exchange mode. The outward and inward pump currents have the same magnitude and time course as short as the membrane capacitance currents indicate that the inward Na ions carry the same amount of ATP hydrolysis energy moving into the cell, which indicate that the kinetic energy carried by the inward Na ions is the driving force for the ATP synthesis occurred in the intracellular loop domain.

The results show that Na/K pump has dual energy convert feature, where kinetic energy plays a significant role in converting the energy.

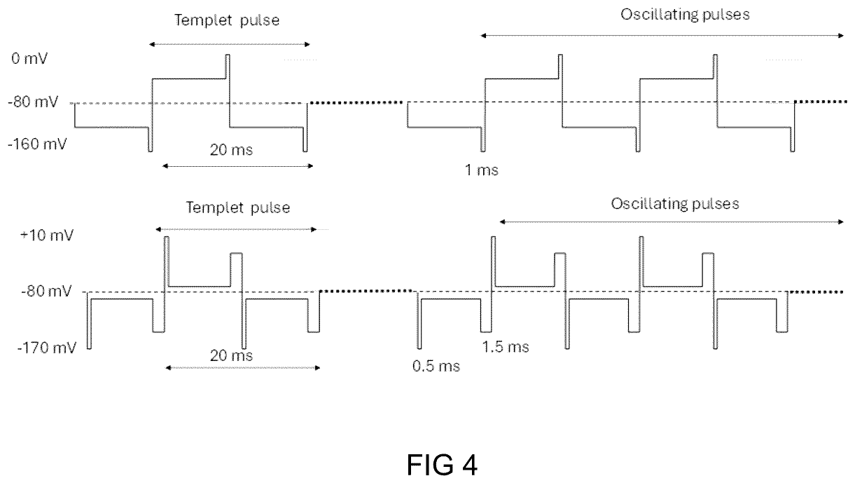

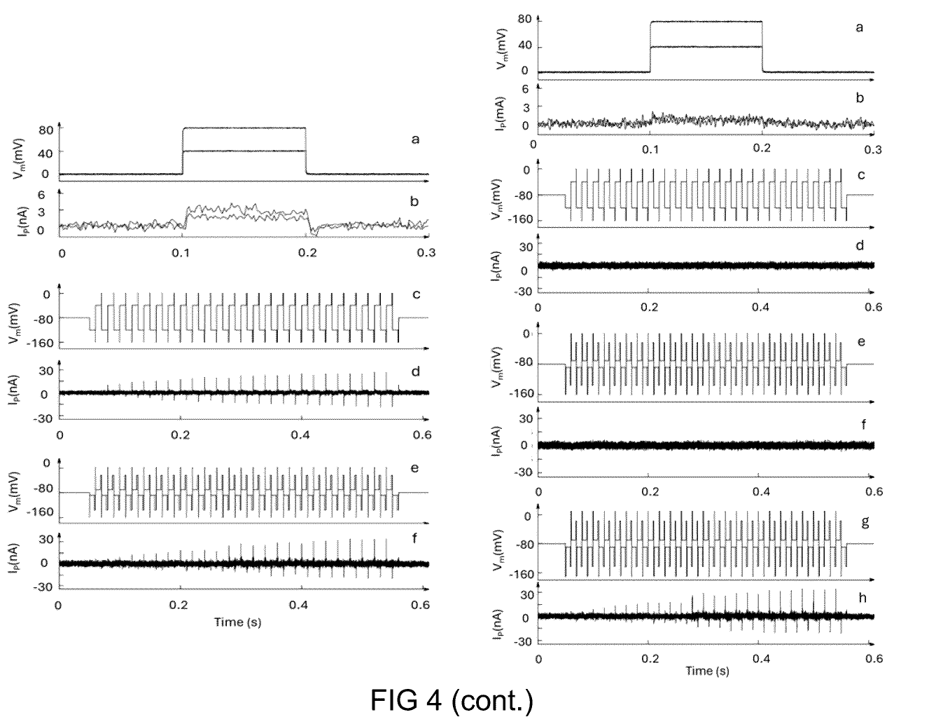

FIG. 4 The upper right panel shows a single pulse of the 2^{nd} generation synchronization electric field, while the upper left is the pre-pulses. Similar in the middle right is the 3^{rd} generation electric field when the left is the corresponding pre-pulse. Lower panel show the pump currents (b) generated by the traditional stimulation pulse (a), the pump currents (d) generated by the 2^{nd} generation electric field (c), and the pump currents (f) generated by the 3^{rd} generation electric field (e) where the magnitude of the activation pulse is 80 mV. The left column represents the presence of ATP while the right column represents after washing out ATP molecules. The oscillating frequency for both the 2^{nd} and 3^{rd} generation electric field is 50 Hz comparable to the natural turnover rate of the pump.

From trace b, we can see that the traditional stimulation pulse-generated pump currents (left) disappeared (right) once the ATP is washout.

Similarly, both the 2^{nd} generation electric field (c) and the 3^{rd} generation electric field with the activation pulse of 80 mV (e) can only generate the pump currents in the present of ATP. Once ATP molecules are washout, either cannot generate pump current (d, f).

However, when the magnitude of activation overshoot is increased to over 90 mV, (g), the pump currents are regenerated regardless of the low level of the ATP concentration. The value is calculation results based on the dynamic model.

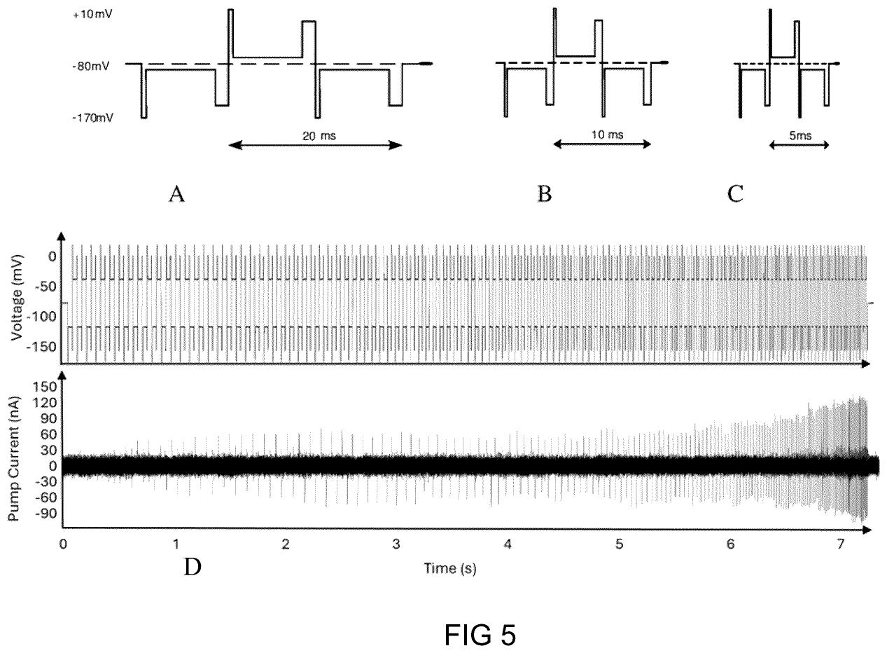

FIG. 5 The upper panel shows three phases of the oscillating electric field: synchronization phase (A) with 50 Hz frequency, modulation phase (B) where the oscillating frequency is gradually increased in a stepwise pattern (3% to 10% of the frequency change for 10 to 20 oscillating pulses) to 150 Hz, and the maintenance phase (C) where the frequency is no longer changed.

The lower panel shows the synchronization modulation electric field generated pump currents. Not only the magnitude of the transient pump currents but also density of the pump currents (the number of transient currents per unit time) are increased.

What is claimed is:

1. A method for controlling Na/K pumps in brain tissue being a specially designed 3^{rd} generation synchronization modulation electric field (3^{rd} generation SMEF) utilizing a device comprising: a synchronization modulation electric field assembly adapted to generate an electric field; wherein the synchronization modulation electric field is distributed throughout a brain.

2. The method of claim 1, wherein a first phase or synchronization phase of the 3^{rd}-SMEF, includes applying an oscillating electric field to synchronize the ion-pumping in half-cycle and the ion-extrusion half-cycle of the Na/K pumps, wherein the ion-pumping in half-cycle of the pumps is in the negative half-cycle and the ion-extrusion half-cycle of the Na/K pumps is in the positive half-cycle of the well-designed oscillating electric field;
applying a negative activation overshoot electric pulse at the start of the negative half-cycle and a negative energy-trap overshoot electric pulse at the end of the negative half-cycle of the oscillating electric field;
applying a positive activation overshoot electric pulse at the start of the positive half-cycle and a positive energy-trap overshoot electric pulse at the end of the positive half-cycle of the oscillating electric field; wherein
application of the negative activation overshoot electric pulse and the negative energy-trap overshoot electric pulse and the electric field plateau in between the negative activation overshoot electric pulse and the negative energy-trap overshoot electric pulse, and the positive activation overshoot electric pulse and the positive energy-trap overshoot electric pulse and the electric field plateau in between the positive activation overshoot electric pulse and the positive energy-trap overshoot electric pulse result in synchronization of the Na/K pumps down to individual steps throughout the pumping cycle; and, wherein
the outward transporter current is restricted at the start of the positive activation overshoot electric pulse in the positive half-cycle and the positive activation overshoot activates the Na-movement to inject electric energy to the Na ions, and the inward transporter currents is restricted at the start of the negative activation overshoot electric pulse in the negative half-cycle of the oscillating electric field and the negative activation overshoot accelerates the K-movement to inject electric energy to the K ions, and synthesize of one ATP molecule during each running cycle of the Na/K pump and the ATP molecule compensates the ATP consumed in actively transporting 3 Na and 2 K ions, and as a result, the ATP net-consumption of the Na/K pumps controlled by the system and method of the present invention is significantly reduced.

3. The method of claim 2, wherein duration of both the positive and negative activation overshoot electric pulses is about 300 μs or less and a magnitude of both the positive and negative activation overshoot electric pulses is at least about 90 mV, where the magnitude is for the field-induced membrane potential in voltage-clamp mode.

4. The method of claim 3, wherein a duration of both the positive and negative energy-trap overshoot electric pulses is about 400 μs or less and magnitude of both the positive and negative energy-trap overshoot electric pulses is about 70 mV, where the magnitude is for the field-induced membrane potential in voltage-clamp mode.

5. The method of claim 4, wherein the modulation phase of the 3^{rd} generation synchronization modulation electric field is to control the pumping rate of Na/K pumps in the brain, adjusting the turnover rate of the Na/K pumps from a physiological value to a predetermined targe value to reduce the metabolic rate of cell in cold water, wherein comprising:
applying an oscillating electric field wherein waveform of the oscillating electric field is identical as the oscillating electric field in the synchronization phase, and the magnitude of the activation overshoot electric pulse and the energy-trap overshoot electric pulse is the same as the magnitude of the activation overshoot electric pulse and the energy-trap overshoot electric pulse in the synchronization phase, respectively, and the duration of the activation overshoot electric pulse and the energy-trap overshoot electric pulse is the same as the duration of the activation overshoot electric pulse and the energy-trap overshoot electric pulse in the synchronization phase, respectively, whereas the duration of the electric plateau is gradually reduced in the modulation phase.

6. The method of claim 5, wherein the oscillating frequency of the electric field in the modulation phase is backward modulation that the oscillating frequency of the electric field is gradually decreased to decelerate the Na/K pumps to a pre-determined target turnover rate.

7. The method of claim 6, wherein the frequency changes in the backward modulation phase are in a stepwise pattern, where the frequency is changed from 3% to 10% and then repeated for approximately 5 to 10 oscillating pulses having the same frequency.

8. The method of claim 7, wherein the last phase, or the maintenance phase of the 3^{rd} generation synchronization modulation electric field is that both the waveform and the frequency of the oscillating electric field are the same as the end of the modulation phase, or the field oscillating frequency remains at the target frequency in the modulation phase.

9. The method of claim 8, where in the 3^{rd} synchronization modulation electric field is applied to brain through a 4-electrode array, wherein four thin and short electrodes are applied to the brain through 4 holes on the skull of the subject. Electric pins are placed in the drilled holes and the 3^{rd} generation SMEF is applied through the whole brain via a series of pin electrodes for maintaining the Na/K activity during ischemic conditions such as stroke;
wherein the pin electrodes are designed as a specific array to be inserted in the skull of the subject; and wherein the four electrodes are computer controlled alternatively into two group, each group consists of two opposite electrodes, the two groups of electrodes are alternatively applied with the electric field, whereby two group of electrodes are never applied with the electric field at the same time and the 3^{rd} synchronization modulation electric field is intermittently applied to the surface of brain, alternatively for every four oscillating pulses.

10. A method of treatment of stroke and related complications in a subject, the method comprising the steps of:
applying a synchronization modulation electric field through a series of pin electrodes designed as a specific array to be inserted in the skull of the subject at predefined locations;
applying the synchronization modulation electric field to a brain with specific methods, including electrode design, a way of applying the electrodes to the brain, and duration of the field application; and
applying the synchronization modulation electric field with optimized parameters of the electric field specifically fit the brain stroke, including magnitude and duration for each section of waveforms, field initial and final frequencies, duration of each phase, and modulation modes.

11. The method of claim 10, comprising maintaining Na/K ATPase activity and a stable delivery of synchronization modulation electric field in the brain.
